(19) United States
(12) Patent Application Publication
(10) Pub. No.: US 2024/0082143 A1
(43) Pub. Date: Mar. 14, 2024

(54) Sunscreen Formulation and Method

(71) Applicant: Beauty Development Corp, Miami, FL (US)

(72) Inventor: Robert Bell, Miami, FL (US)

(21) Appl. No.: 18/466,576

(22) Filed: Sep. 13, 2023

Related U.S. Application Data
(60) Provisional application No. 63/406,162, filed on Sep. 13, 2022.

(51) Int. Cl. A61K 8/9794; A61K 8/27; A61K 8/365; A61K 8/67; A61K 8/92; A61Q 17/04

(52) U.S. Cl. CPC A61K 8/9794; A61K 8/27; A61K 8/365; A61K 8/678; A61K 8/927; A61Q 17/04

(57) ABSTRACT

A sunscreen formulation includes, by weight: 2-4% of rice bran extract; 50-70% of a carrier oil; 10-15% of a wax stabilizer; 18-25% of a mineral sunscreen; and wherein the formulation has a sun protection factor of at least 40. In some embodiments, the mineral sunscreen is zinc oxide, and the rice brand extract together with the zinc oxide provides a sun protection factor of at least 50. In this embodiment, artificial preservatives are not required because the sunscreen formulation is anhydrous.

### CROSS-REFERENCE TO RELATED APPLICATIONS

This application for a utility patent claims the benefit of U.S. Provisional Application No. 63/406,162, filed Sep. 13, 2022.

### BACKGROUND OF THE INVENTION

### Field of the Invention

This invention relates generally to sunscreen formulations, and more particularly to a sunscreen formulation that includes rice bran extract and natural ingredients to achieve a sun protection factor (SPF) of 40 or more.

### Description of Related Art

The prior art teaches a wide range of sunscreen formulations, some of which include natural ingredients, but none of which includes the novel formulation of the present invention.

This invention relates to a sunscreen agent capable of protecting the skin against sunburn upon exposure to ultraviolet radiation in the 2950 to 3150 angstrom unit range, to compositions containing such a sunscreen agent, and to the method of protecting the human skin against burning upon exposure to sunlight.

Erythema or “burning” of the skin which follows excessive exposure to sunlight is caused by ultraviolet rays in the wavelength region between 2950 and 3150 angstrom units (A), while the cosmetically desirable effect of tanning of the skin is due to rays in the wavelength region between about 3150 to 3650 A. Accordingly, a large number of commercial sunscreen compositions are presently marketed in the form of lotions, oils, creams, or the like, which are intended to protect the user against sunburn while permitting tanning to occur. Such commercial compositions contain one or more sunscreen agents which are intended to absorb a substantial portion of the rays in the burning region (2950 to 3150 A) while allowing transmission of a substantial portion of the rays in the region above 3150 A. Numerous commercially prepared chemical compounds, such as salicylates, para-aminobenzoates, napthoates, and the like have been disclosed in the prior art as having the property of absorbing ultraviolet rays in the 2950-3150 A region and therefore capable of being used in such commercial compositions.

However, the ability to absorb rays in the burning region is but one of the many properties a material must possess in order to be suitable for use as a sunscreen agent in commercial compositions. The most essential property is a very high absorption of burning rays so that only very small concentrations of the sunscreen agent need be used in the commercial composition. The agent must also have a high ratio of transmittancy of rays in the tanning region to the transmittancy of rays in the burning region so that tanning may occur. In addition, the material must be stable in sunlight, be capable of absorbing burning rays for a period of several hours, be nontoxic, non-irritating and not have any adverse effects on the skin. Moreover, the sunscreen agent should be soluble in a diversity of solvents, including both oils and non-oily substances, so that it can be readily incorporated in the vehicle or base of the commercial composition, but should have a low degree of solubility in water.

Commercially prepared chemical compounds presently being used as sunscreen agents in commercial compositions generally lack one or more of these properties. For example, salicylates have a low absorptive capacity which therefore requires the use of high concentrations of the order of 10% or more in order to be effective. Para-aminobenzoates have the disadvantage of having an analgesic effect on the skin. Naphthoates have poor solubility characteristics and relatively low absorptivities. Moreover, a substantial number of persons do not desire to use compositions containing commercially prepared chemical compounds and would prefer to use sunscreen compositions containing naturally occurring substances. While some naturally occurring substances such as lanolin, peanut oil, sesame oil and petrolatum have been disclosed as being suitable for use as sunscreen agents, such naturally occurring materials which have been suggested are generally not as effective as chemical sunscreen agents in absorbing high amounts of ultraviolet rays in the burning region.

Ficko, U.S. Pat. No. 10,758,475, for example, teaches a natural, mineral sunscreen that combines natural oxides with high linoleic acid natural oils. The composition may include grape seed oil, safflower oil, zinc oxide, an emulsifier (beeswax), and an antioxidant such as green tea, and other natural ingredients. The composition has an SPF rating of 50+ and provides broad spectrum UVA and UVB protection; however, the formulation does not include rice bran extract, it only includes rice bran wax which is used as an emollient and thickener, not the extract, which functions to increase UV protection.

Ao, U.S. Pat. No. 10,449,138 (Conopco, Unilever), teaches a cosmetic composition comprising film-forming polymer that includes a silicone resin, film forming polymer, beeswax and rice bran wax, optical particle (oleic acid, and/or stearic acid), and at least 20% water by weight of the composition.

Lesniak, U.S. Pat. No. 10,470,999 (Colgate-Palmolive), teaches an anhydrous personal care composition, comprising: (a) 52-53 weight % of caprylic/capric triglycerides; (b) 13.5-15 weight % of a first wax (beeswax) having a melting point of from 62° C. to 64° C.; (c) a second wax having a melting point of 86 to 88° C. present in an amount of 2 weight %; and (d) 10-12.5 weight % of at least one plant oil having a saponification value of from 185 to 195 mg KOH/g. The composition is in the form of a stick, and may include zinc oxide. The patent claims compositions where the second wax is either hydrogenated castor oil or sunflower seed wax, but it does mention rice bran wax as an option.

There are some references that do teach the use of rice bran oil used to increase the SPF of a sunscreen. For example, Loo, U.S. Pat. No. 3,988,436, Plows, U.S. 2022/0110845, Grune, U.S. 2007/0298000, Grune, U.S. 2008/0219938, Von Oppen Bezalel, U.S. 2011/0212040, all teach the use of rice bran oil in a sunscreen formulation. Other references also include mention of rice bran oil as an option for boosting SPF protection and/or as a thickening agent, including Kishida, U.S. 2011/0250250, Loscher, U.S. 2021/0308024, and Eini, U.S. Pat. No. 7,682,623.

### SUMMARY OF THE INVENTION

The present invention teaches certain benefits in construction and use which give rise to the objectives described below.

The present invention provides a sunscreen formulation comprising, by weight: 2-4% of rice bran extract; 50-70% of a carrier oil; 10-15% of a wax stabilizer; 18-25% of a mineral sunscreen; and wherein the formulation has a sun protection factor of at least 40.

In some embodiments, the mineral sunscreen is zinc oxide, and the rice brand extract together with the zinc oxide provides a sun protection factor of at least 50. In this embodiment, artificial preservatives are not required because the sunscreen formulation is anhydrous.

A primary objective of the present invention is to provide a sunscreen formulation having advantages not taught by the prior art.

Another objective is to provide a sunscreen formulation that utilizes rice bran extract to increase the fin protection factor to at least 40, preferably to at least 50 SPF.

A further objective is to provide a sunscreen formulation that is all natural and does not include preservatives.

A further objective is to provide a sunscreen formulation that includes bee pollen, natural vitamins such as vitamin E, and other natural ingredients, while still providing superior protection, stability, waterproofing, and skin protection.

Other features and advantages of the present invention will become apparent from the following more detailed description, taken in conjunction with the accompanying drawings, which illustrate, by way of example, the principles of the invention.

### DETAILED DESCRIPTION OF THE INVENTION

The following specification teaches the invention, a sunscreen formulation and method of protecting skin of a person using the sunscreen formulation, which includes a novel formulation of ingredients including rice bran extract.

Rice bran extract, aka rice bran oil, in a pharmaceutically acceptable carrier, is an effective sunscreen agent, absorbing a very high percent of ultraviolet rays in the burning region (2950-3150 A.). It has an extremely high ratio of transmittancy of rays in the tanning region to the transmittancy of rays in the burning region. Rice bran oil is well suited for use as a sunscreen agent for it has excellent stability upon exposure to sunlight, remains effective for a number of hours, does not discolor or develop odor on exposure to sunlight, is both nontoxic and non-irritating to the skin, and is soluble in oils and common solvents so that it can be compounded and formulated into oils, lotions, creams, and the like for ready application to the skin.

Rice bran oil is obtained from rice bran by conventional expression and solvent extraction techniques well known in the art, using a variety of solvents such as hexane, ether, and the like. Rice bran oil has a relatively low iodine value, a high oleic acid content, is low in saturated fatty acids, and has a low content of fatty acids more unsaturated than linoleic acid.

Rice bran oil is an effective sunscreen agent, absorbing a very high percentage of ultraviolet radiation throughout the burning region (2950-3150 A.). The absorption of burning rays by rice bran oil has been found to be many times greater than other naturally occurring substances which have been suggested heretofore as sunscreen agents, as well as commercial sunscreen compositions containing chemical sunscreen agents. Moreover, rice bran oil has an extremely high differential of greater absorption of burning rays than tanning rays (3150-3650 A.). This absorption differential, which is the ratio of transmittancy of rays in the tanning region to transmittancy of rays in the burning region is many times higher for rice bran oil than for other sunscreen agents or compositions. Such a high absorption differential means that the amount of tanning rays transmitting through the material is high compared to the amount of burning rays transmitted, a characteristic which is extremely desirable in a material intended to be used as a sunscreen agent to prevent burning while permitting tanning to occur.

Rice bran oil, in addition to absorbing a high percent of ultraviolet rays in the burning region and having a very high absorption differential, has a number of other characteristics which make it well suited for use as a sunscreen agent when applied to the skin as such or incorporated in a pharmaceutically acceptable carrier. For example, it is non-toxic and non-irritating to the skin of all subjects tested, has excellent stability upon exposure to sunlight, retains its effectiveness in absorbing burning rays for several hours, and does not discolor, develop odor or otherwise deteriorate upon exposure to sunlight. In addition, for those persons who prefer to use naturally occurring substances rather than commercially prepared chemicals, rice bran oil provides an excellent alternative to the commercial compositions containing such chemicals as the sunscreen agent. Moreover, it can be compounded and formulated with conventional pharmaceutical carriers into oils, lotions, creams and the like for ready application to the skin and is capable of depositing a continuous and durable film on the skin when applied by itself or incorporated in a pharmaceutically acceptable carrier.

The current formulation includes a carrier oil that includes natural vegetable oils comprising sunflower oil and/or jojoba oil, or other suitable natural oil, along with an antioxidant for inhibiting lipid autoxidation. In this embodiment, the antioxidant may be a phenolic antioxidant, such as tocopherol. This may further include ascorbic acid, beta-carotene, and/or Vitamin E.

The formulation may include a stabilizer comprising beeswax and/or other suitable wax. The current formulation may further include a mineral sunscreen such as zinc oxide or titanium dioxide. It is the combination of rice bran extract and mineral sunscreen, along with all natural ingredients, that makes the current formulation superior to the prior art formulations.

It is preferred that the formulation include only a limited number of natural ingredients that provide superior sunscreening capabilities, but which also do not damage the environment. In one embodiment, the formulation consists of a carrier oil, an antioxidant, beeswax, a mineral sunscreen, and rice bran extract.

In one embodiment, the formulation consists of only a natural carrier oil, tocopherol, beeswax, a zinc oxide, and rice bran extract. In the currently preferred embodiment, the formulation consists of about the following percentages of ingredients: 56.9% (all percentages by weight) sunflower seed oil, 5.0% jojoba oil, 0.1% tocopherol, 12.5% beeswax (Cera alba), 3.0% rice bran extract (Oryza sativa), and 22.5% zinc oxide (in combination with polyhydroxystearic acid).

The title of the present application, and the claims presented, do not limit what may be claimed in the future, based upon and supported by the present application. Furthermore, any features shown in any of the drawings may be combined with any features from any other drawings to form an invention which may be claimed.

As used in this application, the words “a,” “an,” and “one” are defined to include one or more of the referenced item unless specifically stated otherwise. The terms “approximately” and “about” are defined to mean+/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A sunscreen formulation comprising, by weight:
2-4% of rice bran extract;
50-70% of a carrier oil;
10-15% of a wax stabilizer;
18-25% of a mineral sunscreen; and
wherein the formulation has a sun protection factor of at least 40.

2. The sunscreen formulation of claim 1, wherein no preservative is included, and the anhydrous nature of the formulation serves as a preservative.

3. The sunscreen formulation of claim 1, wherein the carrier oil is in the form of a combination of sunflower and jojoba oils.

4. The sunscreen formulation of claim 1, wherein the wax stabilizer is in the form of beeswax.

5. The sunscreen formulation of claim 1, wherein the mineral sunscreen is zinc oxide.

6. The sunscreen formulation of claim 1, further including bee pollen.

7. The sunscreen formulation of claim 1, further including an antioxidant.

8. The sunscreen formulation of claim 5, wherein the antioxidant is a phenolic antioxidant.

9. The sunscreen formulation of claim 1, further comprising Vitamin E.

10. A sunscreen formulation comprising, by weight:
50-70% of a mixed vegetable oil;
0.1-2% of tocopherol;
10-15% of beeswax;
2-5% of rice bran extract;
20-25% of zinc oxide in combination with polyhydroxystearic acid;
wherein no preservative is included, and the anhydrous nature of the formulation functions as a preservative; and
wherein the formulation is a lotion and has a sun protection factor of at least 50.
